(19) United States
(12) Patent Application Publication
(10) Pub. No.: US 2023/0293760 A1
(43) Pub. Date: Sep. 21, 2023

(54) Lighting comprising a ventilated disinfection duct

(71) Applicant: EXCLOOSIVA, DARDILLY (FR)

(72) Inventors: Olivier MOYEN, SAINT DIDIER AU MONT D'OR (FR); Hervé PORTRAT, LA GRIGONNAIS (FR); Guillaume GICQUEL, DERVAL (FR)

(21) Appl. No.: 18/018,326

(22) PCT Filed: Jul. 27, 2021
(86) PCT No.: PCT/FR2021/051403

§ 371 Date: Jan. 27, 2023

(30) Foreign Application Priority Data
Jul. 28, 2020 (FR) FR20/07951

(51) Int. Cl. A61L 9/20; F21V 33/00; F24F 8/80; F24F 8/22

(52) U.S. Cl. CPC A61L 9/20; F21V 33/0088; F24F 8/80; F24F 8/22; A61L 2209/12; F21S 8/061

(57) ABSTRACT

The invention relates to a device (1) for lighting and for disinfecting a gaseous mixture (G) comprising: —a first ventilation device (2) designed to suck a gaseous mixture through a suction port (3) of the first ventilation device; —a second ventilation device (4) designed to discharge a gaseous mixture through a discharge port (5) of the second ventilation device; —a disinfection duct (6) connecting the suction port (3) to the discharge port (5) and comprising a plurality of sources of disinfection radiation (7) arranged along the disinfection duct (6), each source of disinfection radiation being configured to emit disinfection radiation intended to disinfect the gaseous mixture (G) sucked into the disinfection duct (6); —a plurality of sources of illuminating radiation (8) arranged along the disinfection duct (6) on an outer surface of the disinfection duct (6) and intended to emit illuminating radiation; and—a housing (9) comprising a plurality of surfaces opaque to the disinfection radiation.

### PREAMBLE

The present invention relates to a device for illuminating and disinfecting a gas mixture.

It is known to produce a device for disinfection of a gaseous mixture by disinfection radiation such as UV rays for example.

It is also known to produce a lighting device intended to illuminate a given room such as a meeting room or a work desk for example.

These provisions are satisfactory in that it is possible to disinfect the ambient air in a room lit by a lighting device.

However, the installation of a disinfection device and an independent lighting device in the same room requires a relatively large location and requires a specific installation process for each device.

The present invention aims to solve all or part of the drawbacks mentioned above.

The technical problem at the basis of the invention consists in particular in providing a device for lighting and disinfection of a gaseous mixture which is of simple and economical structure, while occupying a small space and while being easy to install.

### GENERAL DESCRIPTION

To this end, the subject of the present invention is a lighting and disinfection device according to the aforementioned type comprising:
a first ventilation device arranged to suck in a gaseous mixture through a suction port of said first ventilation device;
a second ventilation device arranged to evacuate a gaseous mixture through an evacuation port of said second ventilation device;
a disinfection conduit connecting the suction port to the evacuation port and comprising a plurality of disinfection radiation sources arranged along the disinfection conduit, each disinfection radiation source being configured to emit disinfection radiation intended to disinfecting the gas mixture sucked into the disinfection conduit;
a plurality of illuminating radiation sources arranged along the disinfection duct on an outer face of said disinfection duct and intended to emit illuminating radiation; and
a box comprising a plurality of faces that are opaque to the disinfection radiation and a face that is transparent or partially transparent to the lighting radiation arranged facing the plurality of sources of lighting radiation.

The gas mixture sucked through the suction port may be infected with a microorganism such as a virus or a bacterium for example, and the gas mixture discharged through the discharge port is disinfected after being exposed to the disinfection radiation in the conduit of disinfection.

The gaseous mixture can for example refer to air.

Advantageously, the lighting and disinfection device saves space compared to an independent decontamination device because the latter must be able to be mounted next to pre-existing lighting systems.

Thus, the lighting and disinfection device is easy to install because it does not require additional space compared to an independent decontamination device.

The arrangements according to the invention make it possible to combine lighting and disinfection functionality in the same device, and thus to save space compared to an independent decontamination device because the latter must be able to be mounted next to pre-existing lighting as well as ease of installation because it does not require additional space compared to an independent decontamination device.

The lighting and disinfection device may additionally have one or more of the following characteristics, taken alone or in combination.

The disinfection duct is substantially cylindrical or polygonal, and for example parallelepipedal and extends along an axis of the duct.

The lighting and disinfection device can be configured in such a way as to maintain an exposure dose inside the disinfection duct with an average value between 1 mJ/cm2 and 7 mJ/cm2, and preferably between 2 mJ/cm2 and 5 mJ/cm2.

The internal volume of the disinfection duct can be defined so as to maintain an exposure dose inside the disinfection duct with an average value between 1 mJ/cm2 and 7 mJ/cm2, and preferably between 2 mJ/cm2 and 5mJ/cm2.

The first ventilation device and the second ventilation device can be configured to provide a volume flow rate of the gas mixture defined so as to maintain an exposure dose inside the disinfection duct with an average value between 1 mJ/cm2 and 7 mJ/cm2, and preferably between 2 mJ/cm2 and 5 mJ/cm2.

A number of disinfection radiation sources of the plurality of disinfection radiation sources can be set so as to maintain an exposure dose inside the disinfection conduit of average value between 1 mJ/cm2 and 7 mJ/cm2, and preferably between 2 mJ/cm2 and 5 mJ/cm2.

Preferably, the axis of the duct is straight.

According to one embodiment, the first ventilation device and the second ventilation device are oriented along the axis of the duct or substantially parallel to the axis of the duct.

In this case, the suction and evacuation of the gaseous mixture takes place parallel to the axis of the duct.

According to one embodiment, the first ventilation device and the second ventilation device are arranged substantially perpendicular to the axis of the duct.

In this case, the suction and evacuation of the gaseous mixture takes place substantially perpendicular to the axis of the duct.

According to one possibility, the first ventilation device and the second ventilation device are embedded in the casing and communicate with the exterior of the lighting and disinfection device through casing openings.

According to one possibility, the box openings comprise a protective wall arranged to allow the suction and the evacuation of the gaseous mixture and to block an evacuation of the disinfection radiation outside the lighting and disinfection device.

The protective wall may be porous and may have a regular polygonal paving structure.

The protective wall can also have a baffle shape, in other words a zigzag shape.

Advantageously, the blocking of the evacuation of the disinfection radiation outside the lighting and disinfection device makes it possible to protect a human from the disinfection radiation which can be harmful.

According to another embodiment, the disinfection conduit may have two bent ends connected respectively to the suction port and to the evacuation port so as to prevent evacuation of the disinfection radiation outside of said device.

The casing may comprise an internal partition arranged to separate the interior of the casing into two hermetic parts, one of the hermetic parts comprising the disinfection duct.

Advantageously, the internal separation of the casing into two hermetic parts makes it possible to limit the risk of contamination of the disinfected gas mixture.

The plurality of illuminating radiation sources may be a plurality of light emitting lamps.

According to one possibility, the plurality of sources of disinfection radiation is a plurality of ultraviolet lamps.

The ultraviolet rays can for example be of the UV-C type and have a wavelength substantially between 100 nm and 280 nm.

Advantageously, ultraviolet rays of the UV-C type can destroy bacteria or viruses whose DNA is sensitive to radiation having wavelengths between 260 nm and 280 nm.

The housing may include suspension rods configured to cooperate with a fixed surface so as to suspend the lighting and disinfection device at a defined height relative to the fixed surface.

The fixed surface can for example designate a ceiling or a floor or a wall of a meeting room.

According to one aspect of the invention, the lighting and disinfection device is configured to produce lighting radiation and disinfection radiation when it is subjected to a DC voltage substantially equal to 12V or 24V.

The plurality of illuminating radiation sources can be configured to have a color rendering index substantially greater than 80.

According to one possibility, the illuminating radiation emitted by the plurality of illuminating radiation sources is configured to have a luminous flux substantially greater than 1000 lumens.

The illuminating radiation emitted by the plurality of illuminating radiation sources may have a color temperature substantially between 2700K and 5000K.

The illuminating radiation emitted from the plurality of illuminating radiation sources may have a beam substantially greater than 30 degrees.

According to one possibility, the lighting and disinfection device is configured to produce a noise level of less than 80 dB.

### DESCRIPTION WITH REFERENCE TO FIGURES

The invention will be better understood with the aid of the detailed description which is set out below with regard to the appended drawings in which:

FIG. 1 is a perspective view of the lighting and disinfection device described according to an embodiment where ventilation devices are oriented along an axis of a disinfection conduit of said lighting and disinfection device.

FIG. 2 is a sectional view of the lighting and disinfection device according to the embodiment described in FIG. 1.

FIG. 3 is a perspective view of the lighting and disinfection device described according to an embodiment where the ventilation devices are arranged substantially perpendicular to the axis of the duct.

FIG. 4 is a sectional view of the lighting and disinfection device according to the embodiment described in FIG. 3.

FIG. 5 is a perspective view of a variant of the lighting and disinfection device shown in FIG. 1, in which the lighting and disinfection device has dimensions substantially greater than those of the lighting device and disinfection of FIG. 1, and wherein the housing of the lighting and disinfection device is cylindrical.

FIG. 6 is a perspective view of the interior of the lighting and disinfection device of FIG. 5.

In the detailed description which will follow of the figures defined above, the same elements or the elements performing identical functions may retain the same references so as to simplify the understanding of the invention.

### Static Description of the Device

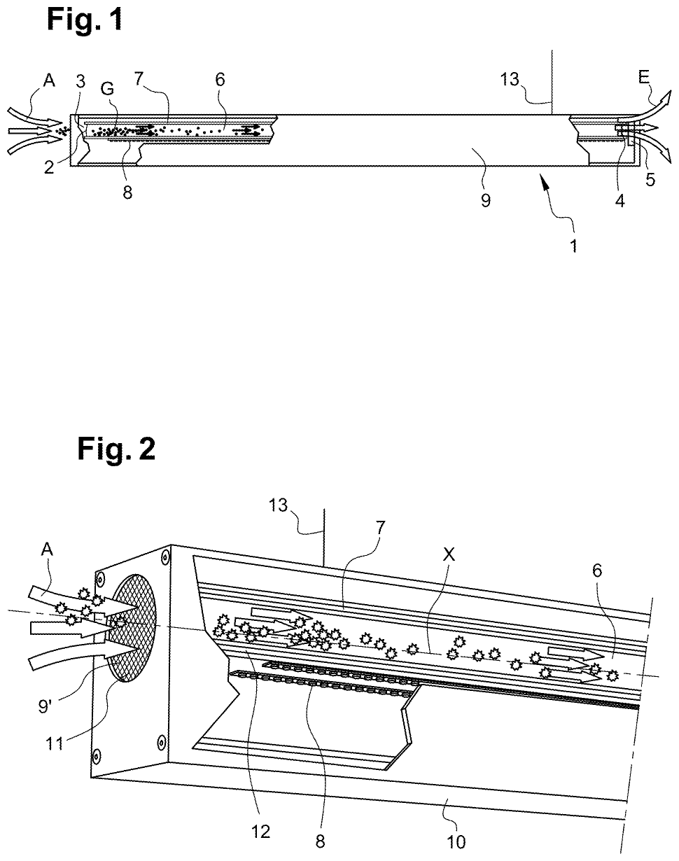

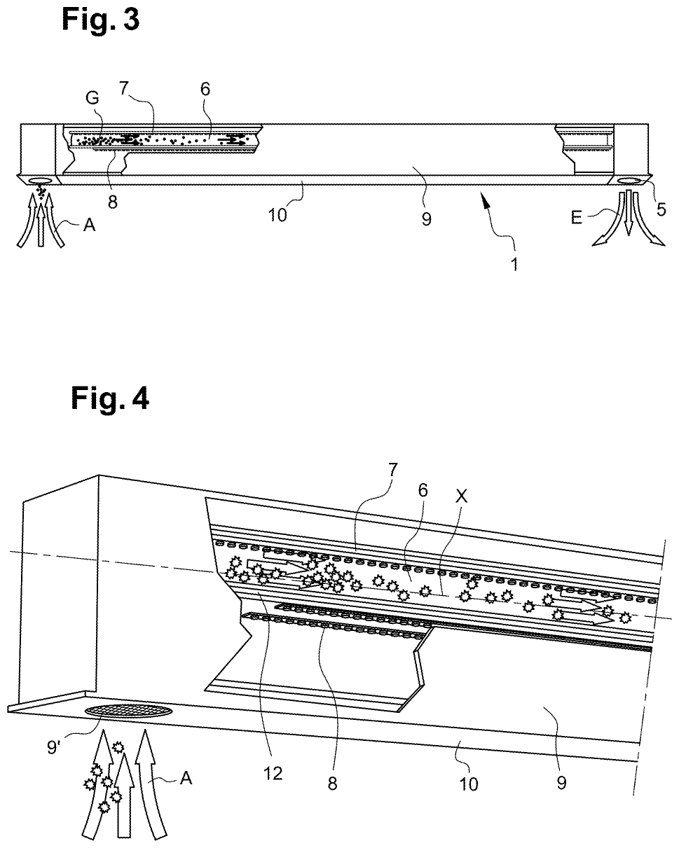

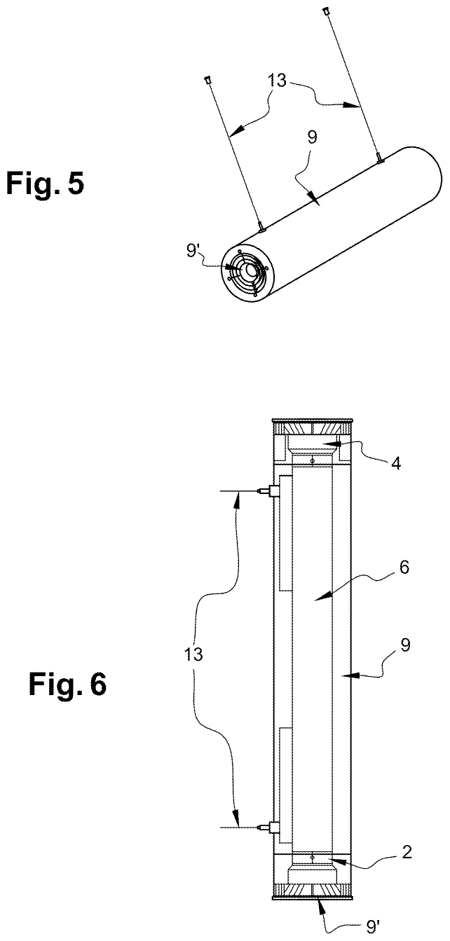

The device 1 for lighting and disinfection of a gas mixture G, shown in FIGS. 1 and 3, comprises a first ventilation device 2 arranged to suck in a gas mixture through a suction port 3 of said first ventilation device, a second ventilation device 4 arranged to evacuate a gaseous mixture through an evacuation port 5 of said second ventilation device, a disinfection conduit 6 connecting the suction port 3 to the evacuation port 5 and comprising a plurality of disinfection radiation sources 7 arranged along the disinfection conduit 6, each disinfection radiation source being configured to emit disinfection radiation intended to disinfect the gaseous mixture G sucked into the disinfection conduit 6, a plurality of radiation sources lighting 8 arranged along the disinfection conduit 6 on an outer face of said disinfection conduit 6 and intended to emit radiation lighting, and a box 9 comprising a plurality of faces opaque to the disinfection radiation and a face

transparent or partially transparent 10 to the illuminating radiation arranged facing the plurality of sources of illuminating radiation 8.

The gaseous mixture can for example refer to air.

The ventilation device may refer to a fan.

Further, the gas mixture sucked through the suction port may be infected with a microorganism such as a virus or bacteria, for example, and the gas mixture discharged through the discharge port is disinfected after being exposed to the disinfection radiation in the disinfection pipe 6.

The disinfection conduit 6 is substantially cylindrical or polygonal, and for example parallelepipedal, and extends along an axis of the conduit X as shown in FIGS. 2 and 4.

The gas mixture G is sucked by the first ventilation device 2 through the first suction port 3 and circulates in the disinfection conduit 6.

During the circulation of the gaseous mixture G in the disinfection conduit 6, particles or microorganisms included in the gaseous mixture G are exposed to electromagnetic radiation from the plurality of disinfection radiation sources 7, for example ultraviolet radiation from UV-C lamps.

An exposure dose designates an amount of energy transmitted by a disinfection radiation source of the plurality of disinfection radiation sources 7 into the gas mixture G per unit area.

The lighting and disinfection device can be configured so as to maintain an exposure dose inside the disinfection conduit 6 with an average value of between 1 mJ/cm2 and 7 mJ/cm2, and preferably between 2 mJ/cm2 and 5 mJ/cm2.

In particular, the lighting and disinfection device can be configured so as to maintain an exposure dose inside the disinfection duct 6 with an average value between 1 mJ/cm2 and 7 mJ/cm2, and preferably between 2 mJ/cm2 and 5 mJ/cm2 regardless of an external environment in which the lighting and disinfection device is installed.

In other words, regardless of a volume of a room in which the lighting and disinfection device is installed, the lighting and disinfection device can be configured so as to maintain an exposure dose inside the disinfection conduit 6 with an average value between 1 mJ/cm2 and 7 mJ/cm2, and preferably between 2 mJ/cm2 and 5 mJ/cm2.

Advantageously, an exposure dose with an average value of between 2 mJ/cm2 and 3 mJ/cm2 allows, for certain types of microorganisms, a disinfection rate of the gas mixture G of between 90% and 99.999%.

According to one possibility, the internal volume of the disinfection conduit 6 is defined so as to maintain an exposure dose inside the disinfection conduit 6 with an average value of between 1 mJ/cm2 and 7 mJ/cm2, and preferably comprised between 2 mJ/cm2 and 5 mJ/cm2.

The first ventilation device 2 and the second ventilation device 4 can be configured to provide a volume flow rate of the gas mixture G defined so as to maintain an exposure dose inside the disinfection conduit 6 with an average value between 1 mJ/cm2 and 7 mJ/cm2, and preferably between 2 mJ/cm2 and 5 mJ/cm2.

For example, the first ventilator 2 and the second ventilator 4 can each provide a volume flow rate between 1 CFM (cubic foot/minute) and 2000 CFM. For example, the first ventilator 2 and the second ventilator 4 can each provide a volume flow of 7.7 CFM.

The first ventilation device 2 and the second ventilation device 4 can be configured to ensure a laminar or turbulent flow of the gaseous mixture G in the disinfection conduit 6.

Advantageously, the turbulent flow of the gas mixture G in the disinfection conduit 6 promotes mixing of the gas mixture G in the disinfection conduit G, and consequently promotes the disinfection of the gas mixture G.

Advantageously, the first ventilation device 2 and second ventilation device 4 allow active circulation of the gas mixture G in the disinfection conduit 6.

The active circulation makes it possible to limit the circulation of the gaseous mixture G in areas weakly lit by the plurality of disinfection radiation sources 7.

A number of disinfection radiation sources of the plurality of disinfection radiation sources 7 can be defined so as to maintain an exposure dose inside the disinfection conduit 6 with an average value between 1 mJ/cm2 and 7 mJ/cm2, and preferably between 2 mJ/cm2 and 5 mJ/cm2.

Advantageously, the lighting and disinfection device can be configured so that the internal volume of the disinfection conduit 6, the flow rate of the first ventilation device 2 and of the second ventilation device 4 and the number of sources of disinfection radiation of the plurality of disinfection radiation sources 7 are adapted to the volume of a room in which the lighting and disinfection device is installed, so as to maintain an exposure dose inside the disinfection with an average value between 1 mJ/cm2 and 7 mJ/cm2, and preferably between 2 mJ/cm2 and 5 mJ/cm2.

The lighting and disinfection device can be installed in a kitchen, a hospital room, a hangar, a shopping center or any other place where one would like to disinfect the air.

Preferably, the axis of the duct X shown in FIGS. 2 and 4 is straight.

According to one possibility, the first ventilation device 2 and the second ventilation device 4 are arranged orthogonally with respect to the axis of the duct X.

The plurality of illuminating radiation sources 8 may be a plurality of light-emitting lamps.

Further, the plurality of illuminating radiation sources 8 is configured to have a color rendering index substantially greater than 80 and the illuminating radiation emitted from the plurality of illuminating radiation sources 8 is configured to have a luminous flux significantly greater than lumens.

Furthermore, the lighting radiation emitted by the plurality of lighting radiation sources 8 has a color temperature substantially between 2700K and 5000K and a beam substantially greater than 30 degrees.

The plurality of sources of disinfection radiation 7, visible in FIGS. 1 to 4, is a plurality of ultraviolet ray lamps and the ultraviolet rays can for example be of the UV-C type and have a wavelength substantially between 100 nm and 280 nm.

Advantageously, ultraviolet rays of the UV-C type can destroy bacteria or viruses whose DNA is sensitive to radiation having wavelengths between 260 nm and 280 nm.

The first ventilation device 2 and also the second ventilation device 4, presented in FIG. 1, are embedded in the box 9 and communicate with the outside of the lighting and disinfection device through box openings 9′ such as the show FIGS. 2 and 4.

The housing openings 9′ include a protective wall 11 shown in FIG. 2 and arranged to allow the suction A and the evacuation E of the gas mixture G and to block an evacuation of the disinfection radiation outside the device. lighting and disinfection.

The protective wall 11 can be porous and can have a regular polygonal paving structure.

The protective wall 11 can also have a baffle shape, in other words a zigzag shape.

Advantageously, the blocking of the evacuation of the disinfection radiation outside the lighting and disinfection device makes it possible to protect a human from the disinfection radiation which can be harmful.

According to an embodiment shown in FIGS. 1 and 2, the first ventilation device 2 and the second ventilation device 4 are oriented along the axis of the duct X or substantially parallel to the axis of the duct X. In this case, the suction A and the evacuation E of the gas mixture G takes place parallel to the axis of the conduit X.

According to one embodiment, the first ventilation device 2 and the second ventilation device 4 are arranged substantially perpendicular to the axis of the duct X. This is the embodiment shown in FIGS. 3 and 4.

In this case, the suction A and the evacuation E of the gaseous mixture takes place substantially perpendicular to the axis of the conduit X and in this case, the disinfection conduit may have two angled ends respectively connected to the suction port 3 and to the evacuation port 5 so as to prevent evacuation of the disinfection radiation outside of said device 1.

The box 9 comprises an internal partition 12 visible in FIGS. 2 and 4 and arranged to separate the interior of the box into two hermetic parts, one of the hermetic parts comprising the disinfection conduit 6.

Advantageously, the internal separation of the casing into two hermetic parts makes it possible to limit the risk of contamination of the disinfected gas mixture.

In the embodiment shown in FIGS. 1 and 2, the housing 9 comprises suspension rods 13 presented configured to cooperate with a fixed surface so as to suspend the lighting and disinfection device at a height defined with respect to the fixed surface.

The fixed surface can for example designate a ceiling or a floor or a wall of a meeting room.

According to one aspect of the invention, the lighting and disinfection device is configured to produce lighting radiation and disinfection radiation when it is subjected to a DC voltage substantially equal to 12V or 24V.

Lighting and disinfection device 1 is configured to produce a noise level of less than 80 dB.

Advantageously, the lighting and disinfection device saves space compared to an independent decontamination device because the latter must be able to be mounted next to pre-existing lighting systems.

Thus, the lighting and disinfection device is easy to install because it does not require additional space compared to an independent decontamination device.

### Description of the Device in Operation

During the operation of the lighting and disinfection device 1 described, the gas mixture G, which can be infected by a microorganism such as a virus or a bacterium for example, is sucked by the first ventilation device 2 through the suction port 3 inside the disinfection duct 6.

The gaseous mixture then passes through the disinfection conduit 6 in which is arranged the plurality of disinfection radiation sources 7 which emit the disinfection radiation, such as UV-C rays for example, intended to disinfect the gaseous mixture G sucked in inside the disinfection duct 6.

Thus the gas mixture G is disinfected all along the disinfection conduit 6 and is subsequently evacuated outside the lighting and disinfection device 1 by the second ventilation device 4 through the evacuation port 5.

Furthermore, the plurality of lighting radiation sources 8 arranged along the disinfection conduit 6 on the outer face of said disinfection conduit 6 emits lighting radiation which passes through the transparent or partially transparent face 10 of the housing 9 and illuminates a given room such as a meeting room or a work office for example.

The two internal parts of the casing, in other words the part comprising the disinfection duct 6 and the part comprising the plurality of lighting radiation sources 8, are hermetically separated by an internal separation wall 12 making it possible to limit the risk of contamination of the gas mixture G disinfected.

The two parts can also operate independently, in other words the lighting and disinfection device 1 described can be used for lighting or for disinfection or both for lighting and disinfection.

Although the invention has been described in conjunction with specific embodiments, it is obvious that it is in no way limited thereto and that it includes all the technical equivalents of the means described as well as their combinations if these fall within the scope of the invention.

1. Device for illuminating and disinfecting a gas mixture-comprising:
a first ventilation device arranged to suck in a gaseous mixture through a suction port of said first ventilation device;
a second ventilation device arranged to evacuate a gaseous mixture through an evacuation port of said second ventilation device;
a disinfection conduit connecting the suction port to the evacuation port and comprising a plurality of disinfection radiation sources arranged along the disinfection conduit, each source of disinfection radiation being configured to emit disinfection radiation intended to disinfect the gaseous mixture sucked into the disinfection conduit;
a plurality of illuminating radiation sources arranged along the disinfection duct on an outer face of said disinfection duct and intended to emit illuminating radiation; and
a box comprising a plurality of faces opaque to the disinfection radiation and a transparent or partially transparent face to the illuminating radiation arranged facing the plurality of sources of illuminating radiation.

2. Lighting and disinfection device according to claim 1, in which the disinfection conduit is substantially cylindrical or polygonal, and extends along an axis of the conduit.

3. Lighting and disinfection device according to claim 1, which is configured in such a way as to maintain an exposure dose inside the disinfection conduit of average value between 1 mJ/cm² and 7 mJ/cm².

4. Lighting and disinfection device according to claim 1, in which the internal volume of the disinfection conduit is defined so as to maintain an exposure dose inside the disinfection conduit with an average value between 1 mJ/cm² and 7 mJ/cm².

5. Lighting and disinfection device according to any one of the claim 1, in which the first ventilation device and the second ventilation device are configured to provide a volume flow rate of the gas mixture defined as so as to maintain an exposure dose inside the disinfection duct with an average value of between 1 mJ/cm² and 7 mJ/cm².

6. Lighting and ventilation device according to claim 1, in which a number of disinfection radiation sources of the plurality of disinfection radiation sources is defined so as to maintain an exposure dose to the inside the disinfection conduit with an average value of between 1 mJ/cm² and 7 mJ/cm².

7. Lighting and disinfection device according to claim 1, in which the first ventilation device and the second ventilation device are embedded in the box and communicate with the outside of the lighting and disinfection device through box openings.

8. Lighting and disinfection device according to claim 7, in which the box openings comprise a protective wall arranged to allow the suction and the evacuation of the gaseous mixture and to block an evacuation of the disinfection radiation outside the lighting and disinfection device.

9. Lighting and disinfection device according to claim 1, in which the casing comprises an internal partition arranged to separate the interior of the casing into two hermetic parts, one of the hermetic parts comprising the disinfection duct.

10. A lighting and disinfection device according to claim 1, wherein the plurality of lighting radiation sources is a plurality of light-emitting lamps.

11. Lighting and disinfection device according to claim 1, in which the plurality of sources of disinfection radiation is a plurality of ultraviolet lamp.

12. Lighting and disinfection device according to claim 1, wherein the box comprises suspension rods configured to cooperate with a fixed surface so as to suspend the lighting device and disinfectant at a defined height in relation to the fixed surface.

13. Lighting and disinfection device according to claim 1, in which the plurality of lighting radiation sources are configured to have a color rendering index substantially greater than 80.

14. Lighting and disinfection device according to claim 1, in which the lighting radiation emitted by the plurality of lighting radiation sources is configured to have a luminous flux substantially greater than 1000 lumens.

15. Lighting and disinfection device according to claim 1, in which the lighting radiation emitted by the plurality of lighting radiation sources has a color temperature substantially between 2700K and 5000K.

16. Illumination and disinfection device according to claim 1, in which the illumination radiation emitted by the plurality of illumination radiation sources has a beam substantially greater than 30 degrees.

17. A lighting and disinfection device according to claim 1, which is configured to produce a noise level of less than 80 dB.
